(19) United States
(12) Patent Application Publication
(10) Pub. No.: US 2014/0364881 A1
(43) Pub. Date: Dec. 11, 2014

(54) COMPRESSION ANASTOMOSIS RING AND SENSOR SYSTEM

(71) Applicant: NOVOGI LTD., Netanya (IL)

(72) Inventors: Gavriel Meron, Petach Tikva (IL); Dror Rosner, Holon (IL); Leonid Monassevitch, Girvat Olga (IL); Jessica Weiss, Tel Aviv (IL)

(21) Appl. No.: 14/363,322

(22) PCT Filed: Dec. 6, 2012
(86) PCT No.: PCT/IB2012/057036

§ 371 Date: Jun. 6, 2014

Related U.S. Application Data
(60) Provisional application No. 61/567,138, filed on Dec. 6, 2011.

(51) Int. Cl. A61B 17/11

(52) U.S. Cl. CPC A61B 17/11; A61B 2562/02
USPC 606/153

(57) ABSTRACT

An anastomosis system for performing anastomosis of a distal lumen with a proximal lumen is described. In one alternative the system includes an anastomosis element having a sensor assembly incorporated therein, the sensor assembly configured to sense the site of anastomosis. In another alternative the system includes an anastomosis component for performing anastomosis of a distal lumen with a proximal lumen; an applicator for applying the anastomosis component; and a sensor assembly connected to the applicator.

### FIELD OF THE INVENTION

The present invention relates to the field of anastomosis. In particular, the present invention relates to an anastomosis device which includes a sensor, for example, an image sensor.

### BACKGROUND OF THE INVENTION

Anastomosis refers to the surgical restoration of the continuity of a hollow organ which has been separated. Excision of a segment of diseased colon or intestine and subsequent anastomosis of the cut end portions is known in the art. Such excision and anastomosis can be carried out by entering the abdominal cavity using either open surgery or a laparoscopic procedure. However, there are significant problems associated with these procedures. The integrity of the anastomosis must be sound so that there is no risk of the anastomosis rupturing or leaking into the abdominal cavity. Opening the bowel lumen and exposing the clean abdominal cavity to contamination increases the risk of postoperative infection.

Stapling may be used for achieving anastomosis of the portions of bowel or intestine to be joined. Using a plurality of staples or other connecting devices, which, of necessity, remain in the bowel, run the risk of leaking or rupture.

Anastomosis rings are used to join severed organ wall portions of a hollow organ; however, the force needed to effect anastomosis is not necessarily constant and is dependent on the thickness of the tissue of the organ to undergo anastomosis.

U.S. Pat. No. 7,527,185 (to Harari et al.) describes a compression anastomosis ring assembly which utilizes a spring element which provides a restorative force. In one embodiment the assembly comprises a first portion which includes an anvil assembly and a second portion which comprises a bottom ring, at least one ring element, and at least one spring element formed of a shape-memory alloy. The at least one spring element provides a restorative force and is in compressive force contact with the bottom ring. The tissue to be joined is positioned between the anvil ring and the bottom ring. A plurality of needles on one of the ring elements are operative, upon application of a closure force, to pierce the tissue and the anvil ring, holding the anvil ring to the second portion of the ring assembly. An applicator for applying the ring assembly and a method for using the assembly and applicator are also described.

The assembly described in U.S. Pat. No. 7,527,185 is an endoluminal anastomosis apparatus for joining preselected organ wall portions of a hollow organ. The apparatus includes a compressive anastomosis ring (CAR) assembly and an endoluminal CAR applicator which allows for endoluminal insertion into organ lumens, including transanal insertion, as well as insertion into small lumens, such as that of the esophagus.

Determining the intra-luminal conditions while performing an endoluminal insertion of an anastomosis device such as anastomosis rings or staples, and obtaining information regarding the anastomosed tissues and anastomosis site may greatly contribute to the success of the insertion and subsequent healing of the surgical site. Endoluminal imaging is possible using endoscopes or other imaging devices, however such devices typically image tissue prior to a procedure. It would be beneficial to view the tissue (both proximal and distal tissues) of an anastomosis site after the anastomosis process has begun.

Information regarding the anastomosis site during and after the anastomosis procedure is not available to-date.

### SUMMARY OF THE INVENTION

The present invention provides an anastomosis system which is capable of imaging (or otherwise sensing) both distal and proximal lumen and also the site of anastomosis before, during and after the anastomosis process.

According to one embodiment the invention provides an anastomosis system for performing anastomosis of a distal lumen with a proximal lumen, the system including an anastomosis element having a sensor assembly incorporated therein, said sensor assembly configured to sense the site of anastomosis.

The anastomosis element may include a ring for compression anastomosis of the distal lumen with the proximal lumen, the ring configured for housing elements of the sensor assembly.

The sensor assembly may include either one or both of an image sensor and a manometer. Other sensors may be included. Typically, a sensor assembly which includes an image sensor may also include an illumination source.

The sensor assembly may further include a transmitter for transmitting in-vivo information.

Anastomosis systems typically include a component that is implanted at the anastomosis site, such as staples or compression anastomosis rings, and an applicator to introduce the implants into the body.

According to one embodiment an anastomosis system includes an anastomosis component for performing anastomosis of a distal lumen with a proximal lumen; an applicator for applying the anastomosis component; and a sensor assembly connected to the applicator.

According to one embodiment the sensor assembly is configured to be shifted from the distal lumen to the proximal lumen after initiation of anastomosis (e.g., after tissues of the distal and proximal lumens have been brought together and are being held in proximity by the anastomosis component).

The anastomosis component may include a top ring and a bottom ring for performing compression anastomosis therebetween. In this case the sensor assembly is configured to be removably disposed in the top ring (the top ring being located within the distal lumen).

According to some embodiments the sensor assembly may include a transmitter for transmitting in-vivo information (e.g., pressure measures, pH values, image data and other in-vivo data).

The system may further include a receiver for receiving information transmitted from the transmitter. The receiver is typically located outside a patient's body. The transmitter may be in wired or wireless communication with the receiver.

The system may further include a display (typically the display is in communication with the receiver) for displaying the information to an operator.

### BRIEF DESCRIPTION OF THE FIGURES

The invention will now be described in relation to certain examples and embodiments with reference to the following illustrative figures so that it may be more fully understood. In the drawings:

FIG. 1 schematically illustrates one embodiment of the system of the present invention;

FIG. 2 shows an exploded view of an applicator according to a first embodiment of the present invention;

FIG. 3 shows an assembled view of an applicator according to a second embodiment of the present invention;

FIG. 4 shows an assembled view of an applicator according to a third embodiment of the present invention;

FIGS. 5a-5c show an anvil assembly according to an embodiment of the present invention in an assembled view (FIG. 5a), in an exploded view (FIG. 5b) and in a cross-sectional side view (FIG. 5c);

FIGS. 6a-c schematically illustrate an applicator of the present invention, according to an embodiment of the invention, positioned at an anastomosis site prior to joining the lumens together (FIG. 6a), after the lumens are joined together, while the anastomosis rings are in contact (FIG. 6b) and as the applicator is pulled in the proximal direction away from the anastomosis site (FIG. 6c); and

FIGS. 7a-b schematically illustrates an implantable component including in-vivo sensors, according to one embodiment of the invention, in cross section (FIG. 7a) and a side view (FIG. 7b).

### DETAILED DESCRIPTION OF THE INVENTION

Generally, embodiments of the present invention provide an anastomosis system which includes an applicator and an implantable anastomosis component. According to one embodiment the system includes a ring applicator, a top ring and a bottom ring for performing anastomosis of a distal lumen with a proximal lumen, and at least one sensor to obtain intra-luminal, in-vivo information and to transmit the information to an operator.

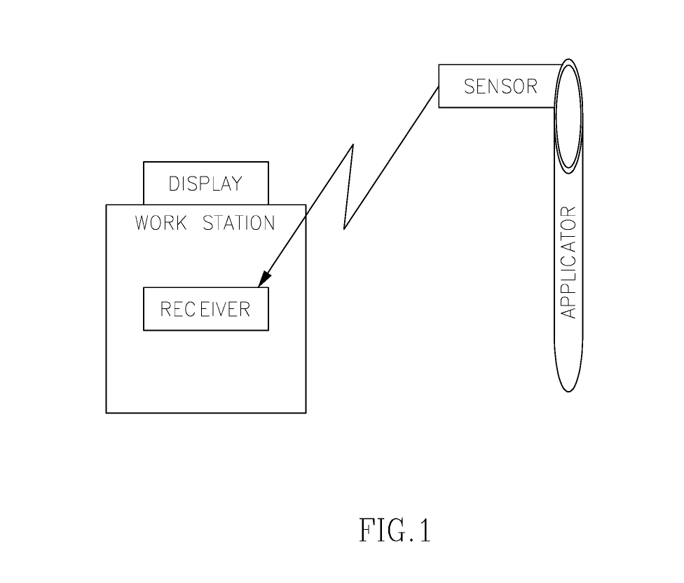

Referring to FIG. 1, an anastomosis ring system according to one embodiment of the present invention is shown schematically, comprising a sensor attached to an applicator which is used for endoluminal insertion of an element such as an anastomosis ring, clip, staple or other element used in anastomosis.

The sensor may be any sensor suitable for obtaining in vivo information such as an image sensor, manometer, temperature sensor, pH meter or oximeter or a combination of sensors. Additional or alternative sensors as known in the art may be used as well. The sensor may be powered by a battery or other suitable power source. At least one sensor may be present, and may include one or a combination of in vivo sensors. The sensor(s) obtains real-time information of the lumen while in the body and transmits the information to a receiver e.g. to a receiver within a workstation. A workstation may include a receiver to receive information from the sensor, a processor to process received information and a display to display information (typically processed information) to the operator. The receiver and monitor are typically located externally to a patient's body.

In one aspect, the sensor communicates with the receiver through a wired connection. Additionally or alternatively, the sensor may communicate wirelessly, e.g., by RF, Bluetooth™ or any other suitable wireless technique.

In one embodiment the applicator and anastomosis ring assembly are similar to the applicator and ring assembly shown and described in U.S. Pat. No. 7,527,185, which is fully incorporated herein by reference.

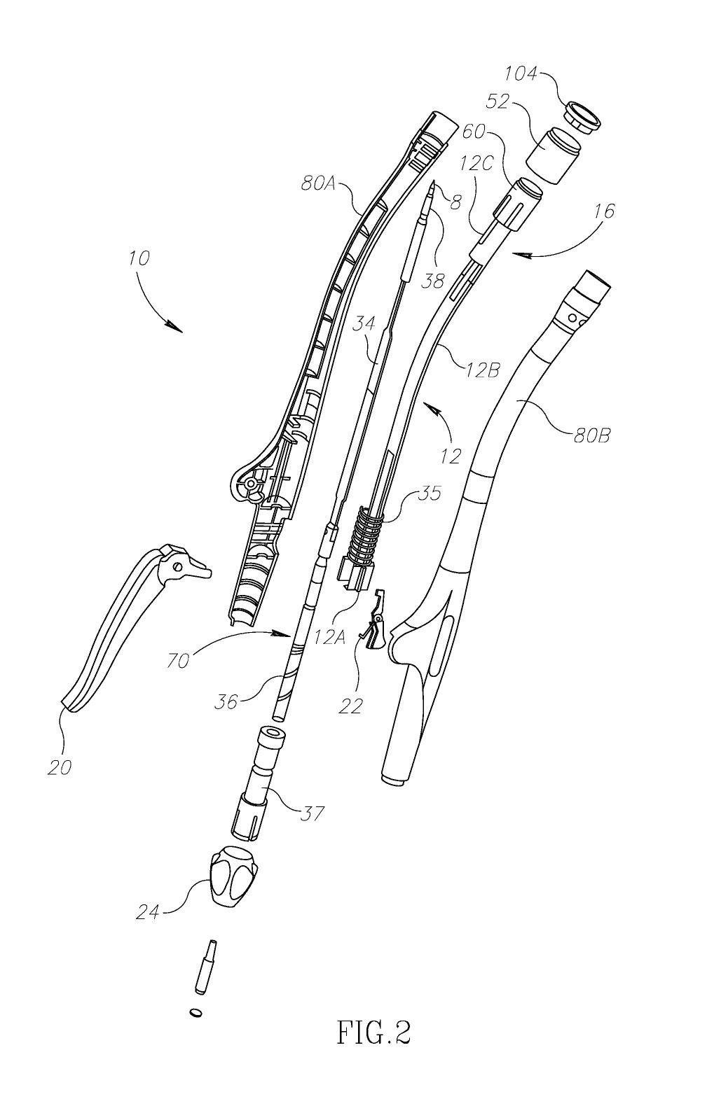

An exploded view of the applicator 10 according to a first embodiment of the present invention is shown in FIG. 2. Applicator 10 is suitable for applying a compression anastomosis ring (also referred to herein below as a “bottom ring”) for use in anastomosis surgical procedures. According to one embodiment the applicator 10 includes a housing, its halves denoted as 80A and 80B. A central member 70 is shown having a control knob 24 positioned at its proximal end and trocar 38 at its distal end. Joined to trocar 38 is trocar connecting link 34 which in turn is in mechanical communication with helix 36 which itself is in mechanical communication with knob shaft 37. Shaft 37 is controlled by control knob 24 which allows for the advance or retraction of trocar 38.

According to one embodiment central member 70 is inserted into a blade pusher assembly 16, which includes a blade pusher 12. Blade pusher 12 has a proximal end 12A connected to its distal end 12C by a linking section 12B. Proximal end 12A of blade pusher 12 is in mechanical communication with a main spring 35. Step slider 60 is positioned at distal end 12C, and is sized and configured for insertion into ring support 52. A bottom ring 104 of the ring assembly (not shown in this figure) is configured and sized to fit onto ring support 52. An anvil assembly (not shown in this figure) which includes an anvil disk is sized and configured to sit on trocar 38 when central member 70 is positioned within blade pusher assembly 16 and when trocar 38 is advanced past distal end 12C of blade pusher assembly 16.

According to one embodiment there is a lever 20 in mechanical communication with proximal end 12A of blade pusher 12. The activation of a cut trigger 22 and the squeezing of lever 20 allow a blade element (not shown) to advance and cut a portion of the tissue held between the anvil disk and bottom ring 104.

According to one embodiment the bottom ring 104 includes a spring element to exert compressive force toward the top ring (not shown) in order to achieve anastomosis. The spring element may include a shape memory alloy or any other suitable elements to exert compressive force.

According to one embodiment a sensor, such as an image sensor 8, having appropriate optics (e.g. lenses, mirrors, etc.) for obtaining an image of the internal walls of the lumen, is attached to trocar 38. According to one embodiment an optical fiber (not shown) runs through central member 70 bringing illumination to the tip of trocar 38 and may be used for transmitting images of the lumen back to an image senor located at a distal end of the central member 70. According to another embodiment the image sensor 8 may include illumination such as LEDs arranged around the image sensor 8 (or otherwise arranged) and a transmitter for wirelessly transmitting images of the lumen to an external receiver.

Image sensor 8 may include a CCD or CMOS image sensor. Small battery powered CCD or CMOS cameras as known in the art may be used.

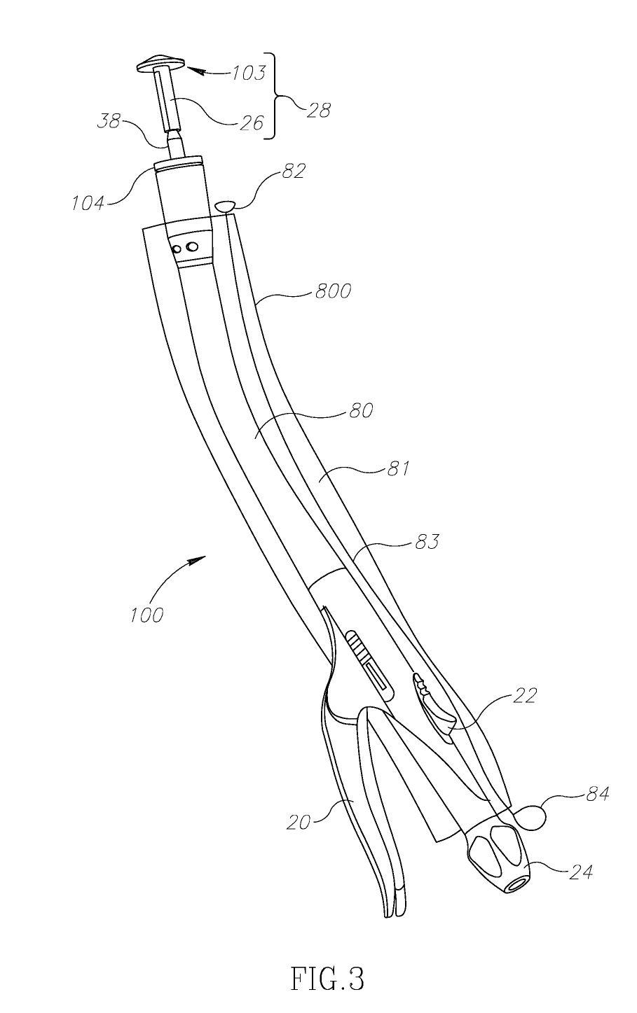

A second embodiment of the applicator 100 of the present invention is shown in FIG. 3 in an assembled perspective view, and comprises all of the essential features of the first embodiment, mutatis mutandis, with the differences described herein below.

Anvil assembly 28 is situated at the distal end of applicator 100, and comprises an anvil disk (or, top ring) 103 and an anvil rod 26. Bottom ring 104 of the ring assembly (comprising anvil disk 103 and bottom ring 104) is affixed directly to the distal end of the applicator 100.

According to the second embodiment, housing 80 of applicator 100 is disposed within a sleeve 800, providing a working channel 81 through which a sensor 82, as well as additional working tools (such as a tool for taking a biopsy, a tool for washing or inflating, and/or other suitable tools) may be passed. Alternatively, the working channel is provided within housing 80 (not shown).

The sensor 82 (or sensor such as sensor 8) may be an image sensor, a manometer or other pressure or proximity sensor, a temperature sensor, a pH meter, an oximeter or another appropriate in vivo sensor. A combination of sensors may be used.

According to one embodiment sensor 82 is mechanically manipulated through one or more cables 83, which are joined with control knob 84 at the proximal end of applicator 100. According to another aspect one or more cables 83 supply power (e.g. electricity) to sensor 82. According to some aspects one or more cables 83 are attached to a receiver (see FIG. 1) outside the patient's body for transmitting information from the sensor.

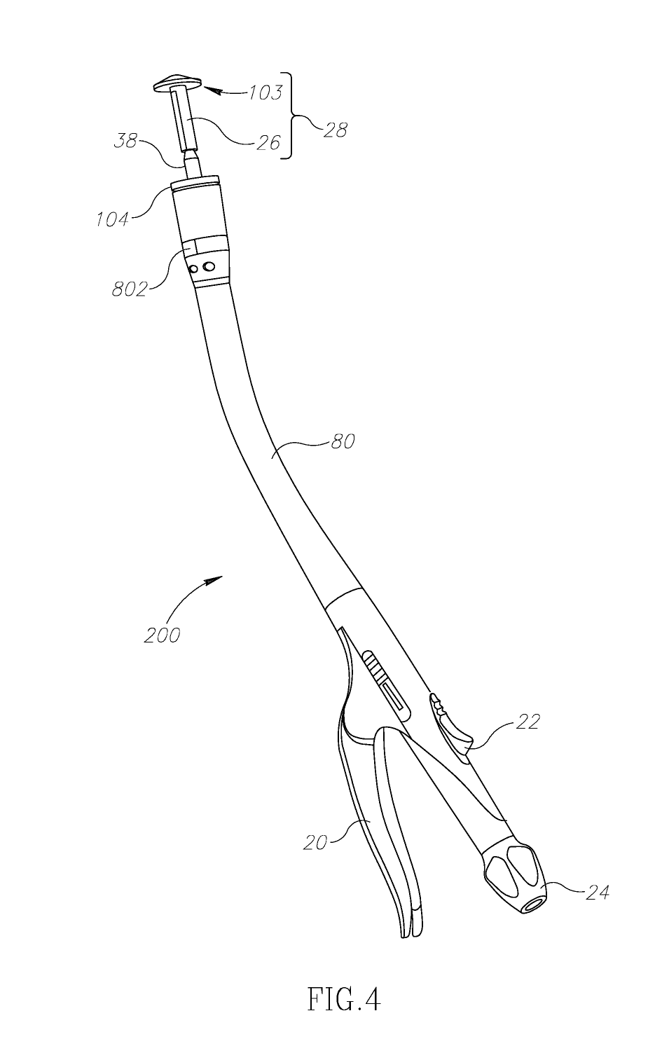

A third embodiment of the applicator 200 of the present invention is shown in FIG. 4 in an assembled perspective view, and comprises all of the essential features of the earlier embodiments, mutatis mutandis, with the differences described herein below.

A wireless battery powered camera 802 or other type of sensor, as described herein above, may be positioned at the distal end of housing 80. Camera 802 may be an integral part of the housing 80 and is preferably flush with the surface of housing 80. Alternatively, camera 802 may be a separate unit that can be attached onto housing 80.

Camera 802 may include its own illumination and optics. For example, LEDs (or other illumination sources) may be arranged around an image sensor such as a CCD or CMOS array. The image sensor and LEDs may be covered by an optical dome to avoid backscatter and otherwise enhance imaging.

Camera 802 is designed to obtain images of the lumen wall as the applicator 200 is inserted in the lumen, and the images are transmitted to a receiver. The camera is capable of taking still pictures or a stream of video.

In some aspects, camera 802 includes an optical system to enable a wide field of view (for example, to obtain a forward looking field of view and/or a side looking field of view).

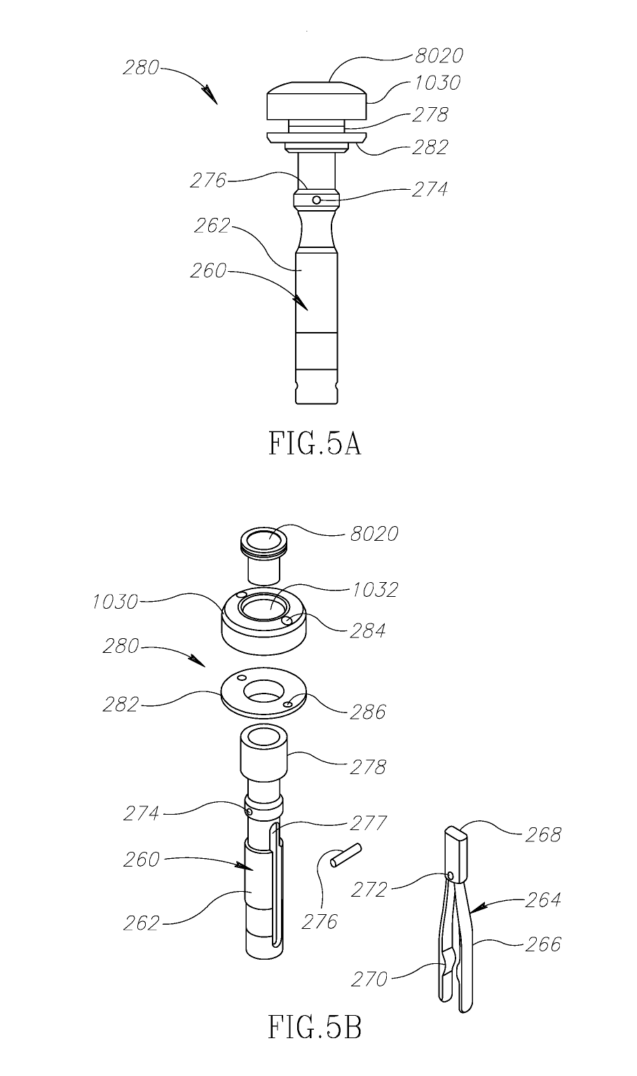

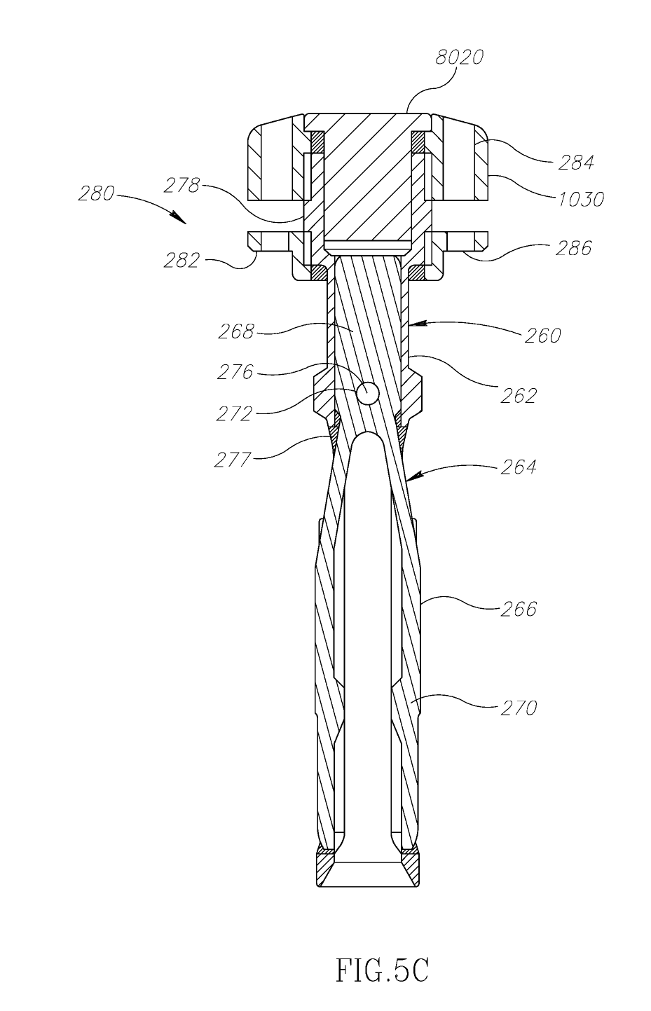

With reference to FIGS. 5a-5c, the anvil assembly 280 situated at the distal end of an applicator (as shown for example in FIG. 2), is shown in FIG. 5a in an assembled view; FIG. 5b shows an exploded view of anvil assembly 280; and FIG. 5c shows a cross-sectional side view of anvil assembly 280 taken along the longitudinal central plane of anvil assembly 280. The applicator comprises all of the essential features of the earlier embodiments, mutatis mutandis, with the differences described herein below.

Still referring to FIGS. 5a-5c, anvil assembly 280 comprises an anvil rod 260 and an anvil disk 1030, wherein a sensor assembly 8020 is removably disposed in the central opening 1032 of the anvil disk 1030 (see FIG. 5b), as described further herein below.

According to one embodiment anvil rod 260 comprises a possibly hollow central body 262 in which a latching member 264 is disposed for receiving the trocar (not shown in this figure) of the applicator. According to one embodiment latching member 264 is a tweezers-like component having longitudinal arms 266 that extend from a head portion 268. Opposing protrusions 270 positioned along arms 266 extend inward for grasping the trocar when coupled therewith. One set of protrusions 270 is shown in the figure, although additional sets and/or alternative configurations may be present. Head portion 268 comprises a through hole 272 which is aligned with through hole 274 in central body 262 of the anvil rod. Latching member 264 may be secured to central body 262 via a pin 276 positioned within the aligned through holes 272 and 274. Typically, the distance between the outer surface of arms 266 is greater than the inner diameter of central body 262, hence arms 266 stick out of opposing longitudinal slots 277 extending along the wall of central body 262. Other suitably designed latching members may be used.

The central body 262 may also include an insufflation tube for insufflating the lumen before or after the anastomosis component is implanted.

According to one embodiment the distal end of anvil rod 260 comprises a cylindrical head 278 having an enlarged diameter for accommodating the sensor assembly 8020 therein. Cylindrical head 278 is shown in the figures as a cylindrical receptacle, however alternative shapes are contemplated depending on design and other considerations.

Anvil disk 1030 is disposed around at least part of the distal portion of cylindrical head 278, and an anchor washer 282 may be disposed around and attached to anvil rod 260 at the proximal portion of cylindrical head 278. Apertures 284 in anvil disk 1030 may be longitudinally aligned with apertures 286 in anchor washer 282 to be joined via pins (not shown) disposed respectively therein.

In one embodiment, sensor assembly 8020 includes an image sensor including optics and illumination. For example, a Video Scout™ camera, comprising a CMOS sensor, quad LED illumination and having a wide field of view may be used, although other suitable image sensors as known in the art may be used.

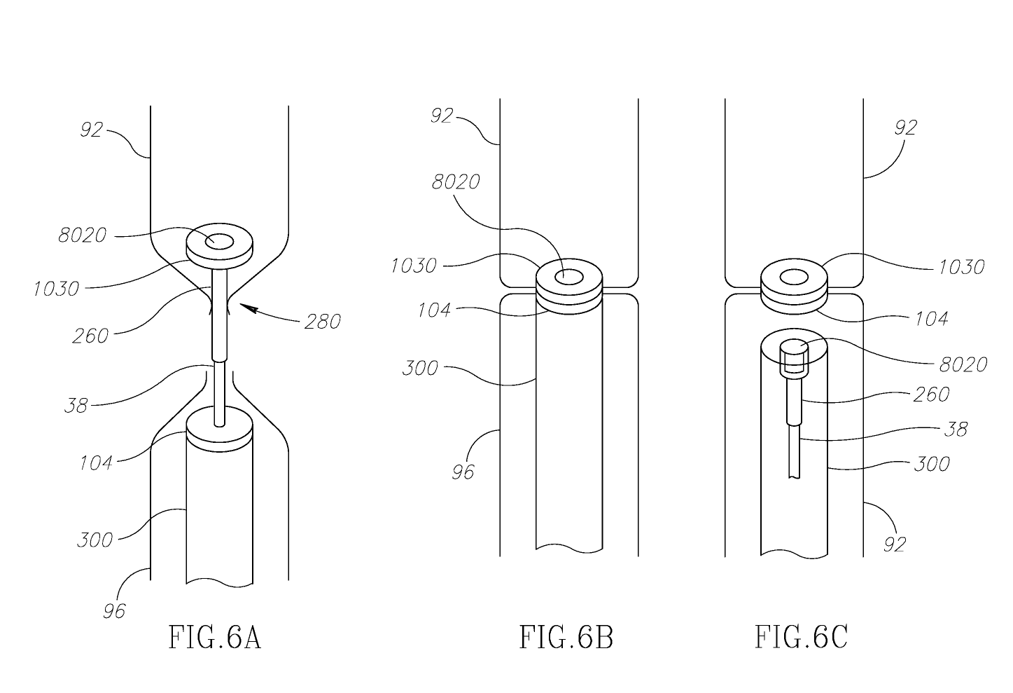

In FIGS. 6a-6c, another embodiment of the present invention is schematically shown, positioned at an anastomosis site prior to joining the lumens together (FIG. 6a), after the lumens are joined together, while the anastomosis rings are brought in proximity to each other (FIG. 6b) and as the applicator is pulled in the proximal direction away from the anastomosis site (FIG. 6c).

In operation, anvil rod 260 and anvil disk 1030 are initially positioned at the distal portion of a dissected intestine (lumen 92), oriented such that sensor assembly 8020 (e.g. a camera assembly) is directed distally, within lumen 92 and anvil rod 260 extends proximally out of the typically loosely sutured lumen 92. Applicator 300 is positioned within proximal portion of dissected intestine (lumen 96), and a bottom ring 104 and trocar 38 extend distally therefrom. Trocar 38 is inserted into and coupled with anvil rod 260, as seen in FIG. 6a.Trocar 38, is pulled proximally, thereby concomitantly pulling anvil assembly 280 until anvil disk 1030 is in contact with bottom ring 104, as seen in FIG. 6b. According to one embodiment the bottom ring 104 comprises a spring element to exert compressive force toward the top ring (anvil disk 1030) in order to achieve anastomosis. The spring element may include a shape-memory alloy.

While positioned within distal lumen 92, the sensor assembly 8020 is able to detect whether potential complications exist in distal lumen 92, such as poor blood supply, as well as whether any tumor remains in distal lumen 92.

A circular blade (not shown) cuts the joined inner ring portions of the proximal and distal lumens 96 and 92, and the joined outer ring portions are held together by anvil disk 1030 and bottom ring 104 until the anastomosis heals.

Referring to FIG. 6c, shortly after the circular blade cuts the lumens 92 and 96, applicator 300 along with trocar 38, anvil assembly 260 and sensor assembly 8020 (shown within applicator 300) are withdrawn from the anastomosis site. As applicator 300 travels in the proximal direction sensor assembly 8020 may view (or otherwise sense) the anastomosis site to ensure anvil disk 1030 and bottom disk 104 are properly positioned, as well as to determine whether potential complications or tumors exist in proximal lumen 96, as described herein above regarding distal lumen 92.

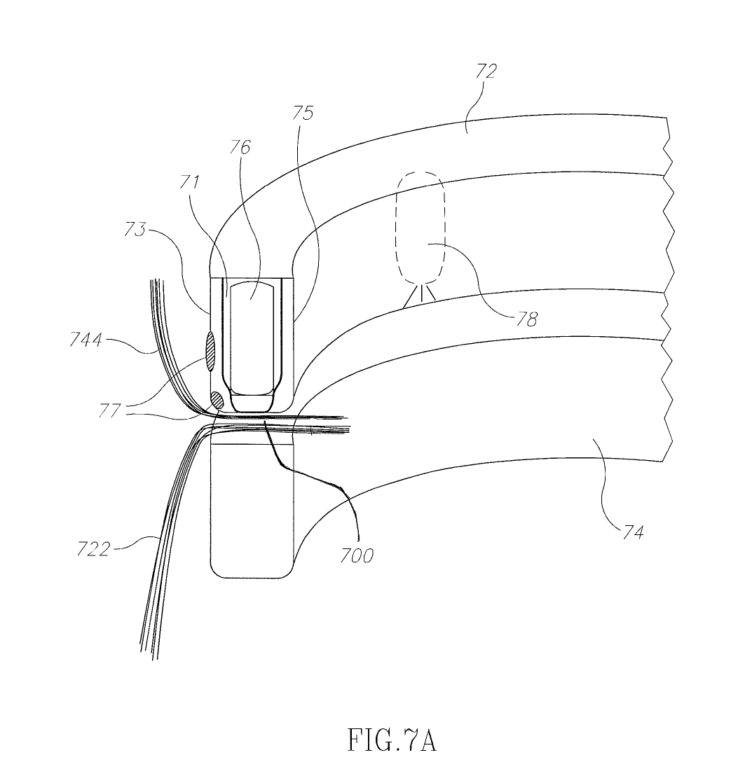

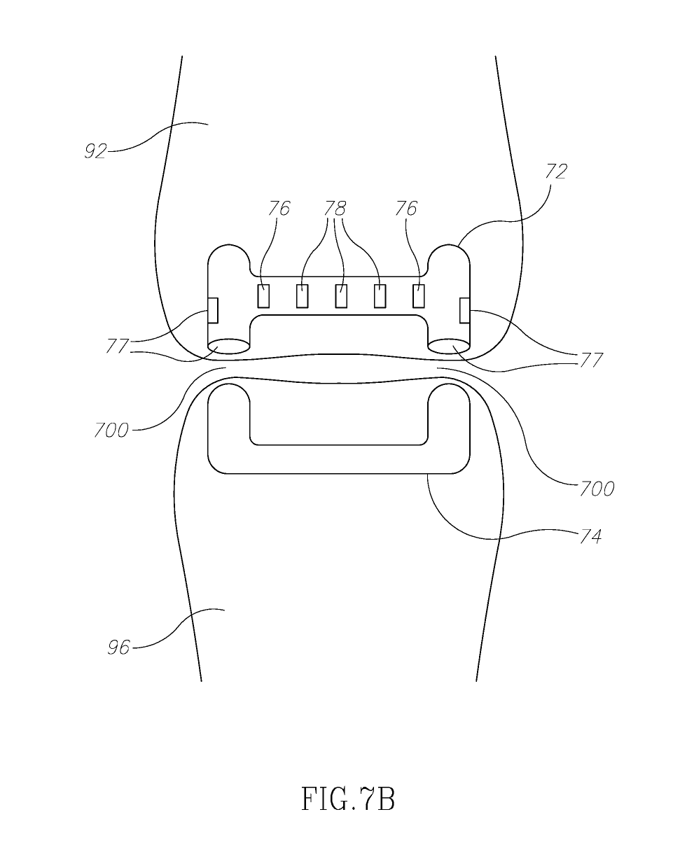

In another embodiment of the invention a sensor may be incorporated within the implantable component. Referring to FIGS. 7a and 7b, a compression anastomosis ring system includes a top ring 72 and a bottom ring 74 for compressing proximal lumen tissue 722 and distal lumen tissue 744. The top ring 722, which may be designed similarly to the design described above, includes an outer diameter wall 73 and an inner diameter wall 75 having a hollow space 71 therebetween. Pins or other elements used to join the top ring 72 and the bottom ring 74 may extend through the top ring 72 into the hollow space 71. The hollow space 71 may house one or more sensors such as camera 76. Additionally, the outer diameter wall 73 and/or the inner diameter wall 75 may house sensors such as manometers 77 (or other pressure or proximity sensors).

Camera 76 may include an image sensor, optics and illumination elements all configured for imaging the anastomosis site 700. Alternatively or in addition, illumination elements (such as LEDs 78) may be placed within the hollow space 71 to provide extra illumination. Camera 76 may include a transmitter for transmitting image information to a receiver (typically located outside of the patient).

Manometers 77 may be designed to sense the pressure at anastomosis site 700 or stress of the tissues 722 and 744. Information may be wirelessly transmitted from manometers 77.

According to some embodiments the bottom ring 74 may also include sensors.

The above-described method for detecting potential complications (such as post-surgery bleeding or defective blood supply to the anastomosis site) or for remnant tumors is a significant improvement over prior art methods, which are typically performed by non-optical methods.

It is understood that the system of the present invention can comprise any one or all of the sensors described herein above.

It is further understood that the above description of the embodiments of the present invention are for illustrative purposes only, and is not meant to be exhaustive or to limit the invention to the precise form or forms disclosed, as many modifications and variations are possible. Such modifications and variations are intended to be included within the scope of the present invention as defined by the accompanying claims.

1. An anastomosis system for performing anastomosis of a distal lumen with a proximal lumen, said system comprising an anastomosis element having a sensor assembly incorporated therein, said sensor assembly configured to sense the site of anastomosis.

2. The system of claim 1 wherein the anastomosis element comprises a ring for compression anastomosis of the distal lumen with the proximal lumen, said ring configured for housing elements of the sensor assembly.

3. The system of claim 1 wherein the sensor assembly comprises either one or both of an image sensor and a manometer.

4. The system of claim 3 wherein the sensor assembly comprises an illumination source.

5. The system of claim 1 wherein the sensor assembly comprises a transmitter for transmitting in-vivo information.

6. An anastomosis system comprising:
an anastomosis component for performing anastomosis of a distal lumen with a proximal lumen;
an applicator for applying the anastomosis component; and
a sensor assembly connected to the applicator.

7. The system of claim 6 wherein the anastomosis component comprises a top ring and a bottom ring for performing compression anastomosis therebetween.

8. The system of claim 7, wherein the sensor assembly is configured to be removably disposed in the top ring, wherein said top ring is located within the distal lumen.

9. The system of claim 6, wherein the sensor assembly is configured to be shifted from the distal lumen to the proximal lumen after initiation of anastomosis.

10. The system of claim 6 wherein the sensor assembly comprises a transmitter for transmitting in-vivo information.

11. The system of claim 10, further comprising a receiver for receiving information transmitted from the transmitter, wherein the receiver is located outside a patient's body.

12. The system of claim 11, wherein the transmitter is in wired communication with the receiver.

13. The system of claim 11, wherein the transmitter is in wireless communication with the receiver.

14. The system of claim 11, further comprising a display for displaying the information to an operator, wherein the display is in communication with the receiver.

15. The system of claim 7, wherein the bottom ring comprises a spring element to exert compressive force toward the top ring in order to achieve anastomosis.

16. The system of claim 15, wherein the spring element comprises a shape-memory alloy.

17. The system of claim 6, wherein the sensor assembly comprises one or more or a combination of an image sensor, manometer, temperature sensor, pH meter and oximeter.
